(19) United States
(12) Patent Application Publication
(10) Pub. No.: US 2020/0338348 A1
(43) Pub. Date: Oct. 29, 2020

(54) Multimodal Transcutaneous Auricular Stimulation System Including Methods and Apparatus for Self Treatment, Feedback Collection and Remote Therapist Control

(71) Applicants: Jonathan M. Honeycutt, Holiday, CA (US); Thomas Anthony La Rovere, Santa Ynez, CA (US)

(72) Inventors: Jonathan M. Honeycutt, Holiday, CA (US); Thomas Anthony La Rovere, Santa Ynez, CA (US)

(21) Appl. No.: 16/153,800

(22) Filed: Oct. 7, 2018

Related U.S. Application Data
(60) Provisional application No. 62/569,588, filed on Oct. 8, 2017.
(60) Provisional application No. 62/733,903, filed on Sep. 20, 2018.

(51) Int. Cl. A61N 1/36; A61N 1/02; A61N 5/06; A61M 21/02; A61N 1/04; A61B 5/00; A61B 5/0205; A61B 5/145; A61B 5/0476; G16H 20/30; G16H 40/67

(52) U.S. Cl. CPC A61N 1/36036; A61M 2021/0027; A61N 1/36034; A61N 1/025; A61N 5/0622; A61M 21/02; A61N 1/0484; A61B 5/486; A61B 5/0022; A61B 5/0205; A61B 5/14542; A61B 5/0476; A61B 5/4035; A61N 1/36031; A61N 1/0456; G16H 20/30; G16H 40/67; A61N 1/36014

(57) ABSTRACT

A modular, multi-modal energy therapy system for electrical and electromagnetic stimulation includes signal generating, conditioning, and control electronics, stimulation monitoring electronics, signal conduits, and wearable energy emitter modules configured for coupling energy emitters to surfaces of the human ear for transcutaneous energy delivery to nerves in the auricular nerve field. Electrical emitter modules configured with electrodes deliver electrical stimulation; electromagnetic emitter modules configured with light emitting diodes deliver electromagnetic stimulation. A computer controls signal generating electronics and provides internet connectivity with a remote server. Application software includes stimulation programming and parameter selection, and databases containing user data, records of stimulation sessions, user responses to symptom assessment instruments, and biofeedback sensor input enable local and remote monitoring of a user's health status by therapists.

### CROSS-REFERENCE TO RELATED APPLICATIONS

Application No. 62/733,903, A Multimodal, Modular Transcutaneous Auricular Stimulation System Including Methods and Apparatus for Self-Treatment, Feedback Collection and Remote Therapist Control

Application No. 62/569,588, Neurostimulation Therapy System Including Methods and Apparatus for Administration, Feedback Collection and Remote Control

### BACKGROUND

The advent of mobile technologies is rapidly changing the modus operandi of modern medicine, connecting the private lives of healthcare recipients to online monitoring systems accessible to their nurses, therapists and physicians on a continuous basis. Robotic therapist algorithms may soon instantly sense and respond to changes in the biopsychological and behavioral data received from user-worn sensors. Robotic therapists or algorithmic monitoring may be configured to send alarms and status updates to healthcare providers whenever a patient response parameters exceed or fall below selected thresholds. For the first time in history, it is possible to permanently connect doctors and patients and deliver dynamically monitored, data-driven electromedicines and electromagnetic therapies with full transparency, clinical responsivity and, most importantly, patient accountability. We stand at the threshold of a new age of stimulus-response-driven electro- and electromagnetic medicine, which comprise the purpose, means and methodology of the present invention.

### FIELDS OF INVENTION

The present invention comes from the emerging fields of neurostimulation and computerized health, wellness and medical therapeutics. Neurostimulation may be broadly defined as the application of electrical or electromagnetic energy to nerves targeted directly or indirectly (e.g., by applying energy to surrounding, connecting and/or conductive tissues, e.g., skin, tissues and vasculature), for the purpose of producing beneficial changes in the activity of neurotransmitters, the activity within structures and centers of the brain, in neuronal and synaptic activity, and as streams of cascading effect producing changes in the activity of organs, especially organs in communication with the autonomic nervous system, within the nervous system, especially the autonomic nervous system.

Neuromodulation is unique among health, wellness and medical interventions and therapeutic modalities because the intervention involves applying electrical or electromagnetic energy to certain areas of the human body, it can be dispensed electronically. Its electronic delivery to a user may be controlled by a computer locally or remotely, through a computer communications network.

The present invention exemplifies the merger of neurostimulation therapy with computerized health, wellness and medical therapeutics by incorporating, as a system and a method of use, a computerized, network-based system capable of electronically delivering neurostimulation interventions and performing the measurement, recordation and transmission of data, through communication networks, logging the delivered neurostimulation intervention and its effects on a user, to enable real-time remote monitoring and dynamic interventional adjustment by remote health, wellness and medical personnel.

A key component of this system is an electronically controllable, electronically deliverable, electronically recordable therapeutic modality which we define as neuromodulation, also known as “neurostimulation.” Hereinafter, the terms “neuromodulation,” “neuro-stimulation,” “neurotherapy” and “nerve stimulation” are used interchangeably and refer to the full range of therapeutic modalities and methods by which energy, as electricity and, in the is future, electromagnetic energy, may be electronically delivered to anatomical structures of a mammalian body, such structures including nerves, tissues including connective tissues, organs individually and systemically, muscles, vasculature and glands. It will also refer to devices designed and used to accomplish such energy delivery through direct or indirect application to said anatomic structures, using external, non-invasive, transcutaneous means and minimally invasive percutaneous means.

The present disclosure describes a computer-controlled, cloud-connected, network-integrated, remotely-monitored, supervised and managed therapeutic system incorporating the aforementioned methods and devices employing non-invasive and minimally invasive means to generate, deliver and apply energy stimulation to the aforementioned anatomic structures of users, with particularity to nerves and nerve tissues, muscles and muscle tissues, vasculature and vascular tissues, and skin and skin tissues, to provide a wide variety of therapeutic effects and health benefits, including such effects and benefits as may be realized according to the anatomic location, biological operation, and systemic functionality of one or more selected stimulation targets.

Using electrical stimulation to modify or “modulate” the activity of nerves, neurotransmitters, neurons, synapses and selected centers of the brain and the nervous system is known and defined among practitioners as neurostimulation, neuromodulation, neurotherapy or simply “nerve stim,” which will be used interchangeably herein along with “electrostimulation.” A well-known use of transcutaneous electrical nerve stimulation is pain control and relief, commonly referred to by its acronym, “TENS”. Beyond pain relief applications, neurostimulation devices are also known to produce a wide variety of additional therapeutic benefits using invasive measures. Such invasive methods require the surgical implantation of a pulse generator and an electrode or conductive wire into the body where it is wrapped around the cervical branch of the vagus nerve.

Invasive stimulation of the vagus nerve via stimulator implant has been successfully used for at least twenty years and has been approved by the FDA for the treatment of epilepsy and is treatment resistant depression, and is under intensive study to treat other conditions such as anxiety, insomnia, migraine, weight loss, management of pain, obesity, and Alzheimer's disease, and Parkinson's disease, among others. The advantages of using implantable stimulation devices is their constant, wired connection to the nerve, long life batteries, and their resistance to tampering by the patient (when the signal generator is implanted under the skin) by virtue of their effective inaccessibility to the patient. Disadvantages of invasive nerve stimulation devices include their cost, the expense of surgical implantation, the need for follow-on surgeries to change batteries and replace faulty or outdated nerve stimulator electronics, complications of wound care, the risks and dangers of surgery including infections, nerve damage, anesthesia risks and the patient's lack of control over the stimulator and dependence on expensive physician intervention.

Another class of invasive nerve stimulation devices includes those classified as “minimally invasive” which incorporate a needle or an array of needle electrodes which pierce and penetrate the skin to produce percutaneous nerve stimulation. U.S. Pat. No. 9,662,269 B2 describes a recent variant of these percutaneous nerve stimulation devices. Another example of such a system is disclosed in U.S. Patent Publication No. 2013/0150923, reflecting a device sold by Biegler GmbH under the trade name P-STIM®. A significant drawback of such systems is that the needle electrodes break the skin, causing pain and the consequent patient aversion, as well as the risk of infection. Additional disadvantages of such semi-invasive percutaneous stimulation devices include their relatively high cost, the expense of surgically implanting skin piercing percutaneous needle electrodes, the need for follow-on surgeries to re-position needle arrays, complications of wound care, the risks and dangers of surgery including infections, nerve damage, the pain of percutaneous needle puncture, and the patient's lack of control over the stimulato the fact that it can only be done by specially trained medical professionals.

Clinical research on animals and human beings has demonstrated that electrical stimulation of the vagus nerve via the auricular nerve field produces identifiable and recordable activity in various centers of the brain, including, inter alia, the brains control nexus, the nucleus of the solitary tract (NST) and the accumbens nucleus, known as the “reward center” of the brain. The accumbens nucleus is the location wherein synaptic activity is known to modulate is various forms of stimulus and reward seeking behavior associated with addiction and self-control in activities from eating and sex to drug and alcohol use. Most recently, the neuromodulation of activity in the accumbens nucleus was demonstrated using functional MRI (fMRI) during transcutaneous electrical stimulation of the auricular nerve field. Specifically, Cymba conchae stimulation, compared to earlobe (control) stimulation, produced significant activation of the “classical” central vagal projections, e.g., widespread activity in the ipsilateral nucleus of the solitary tract (NST), bilateral spinal trigeminal nucleus, dorsal raphe, locus coeruleus, and contralateral parabrachial area, amygdala, and accumbens nucleus. Bilateral activation of the paracentral lobule was also observed. Deactivations were observed bilaterally in the hippocampus and hypothalamus. These findings provide evidence in humans that the central projections of the ABVN are consistent with the “classical” central vagal projections and can be accessed non-invasively via stimulation of the human auricle.

Four primary sensory nerves area found in the externally projected dimensions of the human ear: the auriculotemporal nerve; a branch (v3) of the trigeminal nerve; the great auricular nerve; the auricular branch of the vagus nerve; and the lessor occipital nerve. A transcutaneous method of nerve stimulation may target one or more of these nerves in the auricular nerve field singly or in combination, including the auricular branch of the vagus nerve in the tragus; concha cava and cymba; the great auricular nerve in the ear lobe; the lesser occipital nerve around the medial and inferior helix, and the auriculotemporal nerve in the triangular and scapha fossa and the legs of antihelix just below the round crest of the ear. The auriculotemporal nerve is the largest of the three divisions of the trigeminal nerve, the fifth cranial nerve (CN V), and is also known as the mandibular nerve (v3). Applying an electrical stimulus to the left cymba conchae using a stimulus intensity close to or above the sensory detection threshold, but below the pain threshold results in neuronal, synaptic and brain activation patterns similar to that of left cervical vagus nerve stimulation using an implanted stimulator.

The present invention includes methods for controlling, selecting and optimizing the intensity, pulse duration, frequency and other electrical characteristics of neuromodulation to conduct electricity into the thick, myelinated Aβ fibres of the auricular branch of the vagus nerve (ABVN). These nerve fibers, like those of the cervical branch of the vagus nerve, project is directly to the nucleus of the solitary tract (NST) in the brainstem. The NST serves as a nexus to activate and potentially modulate a complex interconnected cerebral network. Reaching the nucleus of the solitary tract (NST), transcutaneous nerve stimulation of the ABVN produces effects which closely correspond to those produced by invasive vagus nerve stimulation using implanted and percutaneous stimulators, including the ability to produce anti-convulsive effects for which invasive electrical vagus nerve stimulation was originally developed.

Although numerous studies support the efficacy of transcutaneous vagus nerve stimulation for a variety of clinical indications, further research is needed to established clinical paradigms and protocols for stimulation parameters such as duration and frequency of each stimulation session, length of treatment; electrical frequencies to be used, pulse-widths, waveforms, nerve targets, and electrode placements, etc.

The present disclosure relates to methods of applying energy stimulation to anatomic structures such as nerves, skin tissues, muscles, connective tissues, and devices used to accomplish such stimulation using modulated energy emissions applied directly to human tissue through external, non-invasive measures. Disclosed is a system and methods for comprising an intelligent, stimulus-response based energy stimulation therapy system that delivers energy stimulation to the nerve targets of a user, collects user response feedback as subjective self-assessment responses to empirically developed symptom-sampling scales and as biofeedback obtained from user-worn sensors, wherein energy stimulation parameters and protocols may be adjusted and calibrated for maximal effectiveness by a remote therapist or an algorithm according to said user “Iometrics” or user generated data. As the energy used to produce nerve stimulation, the system may employs an energy emitter module which is configured as traditional electrodes delivering electrical energy, and or alternatively integrate emitters of electromagnetic energy emitting wavelengths in both the visible green, blue and red wavelengths to the invisible fields of infrared.

### PRIOR ART

As the nerve of the auricular nerve field lay a mere 1 to 3 millimeters below the skin surface of certain ear locations, such as the concha and concha cymba, this area provides ideal targets for transcutaneous electrical nerve stimulation of the vagus nerve. Despite this anatomical access to the vagus nerve, there are a number of challenges in designing the interface coupling the stimulation device to the target area of human tissue using a coupler containing electrodes which transmit the stimulation signal transcutaneously across the skin to the targeted nerve field. The two most significant of these challenges are the secure attachment of the electrode-skin coupler containing the electrode and the comfort of said coupler according to a user.

Cerbomed GmbH manufactures a transcutaneous tVNS device (NEMOS®), with a handheld controller connected by wire to an earpiece that wedges two metal electrodes, one anode and one cathode, against the skin of the concha cymba of the ear at two points between 5 and 12 millimeters apart. This scaffold-like coupler earpiece retains the position of the electrodes and maintains the constant contact forces of the electrodes against the skin via spring forces created between superior and inferior anchor-points, with a lower “earbud-like” component positioned inferiorly in the lower concha and a superior component wedged under the superior ridge OD the conch cyma. Positional retention of this ear piece relies on constant spring forces which are adjusted by the user using a sliding mechanism on the lower part of the scaffolding. As there is little subcutaneous padding in the skin proximal to the superior and inferior contact points on the ear, the spring force required for position retention and the electrode contact maintenance may be poorly tolerated over prolonged periods of treatment. The NEMOS®, scaffold electrode would be unsuitable for wearing during sleep. Additionally, this coupling scheme is limited to a single position which may be sub-optimal for many users, as the auricular nerve matrix and tissue architecture of the ear can vary significantly from one individual to another. Hence, the Nemos® lacks the flexibility to work effectively when other electrode positions are required, as with those who not possess a matching combination of nerve matrix and ear structure. This electrode location limitation does not accommodate variances in nerve field receptivity associated with normal anatomical variations in the ears of different individual users. The attachment security of the Nemos® earpiece is maintained by anchoring it in the inferior portion of the conchal bowl with a hollow circular piece of plastic that partially or completely occludes access to the ear canal. This means that the gravity drag of the cable weight and any additional gravity or sheering forces that may be suddenly applied to the cable, for example by snagging the cable on a table corner or any one of thousands of other snag-risks, or by dropping the handheld controller, are immediately transferred to the anchor sitting in the conchal bowl, and thereby to the concha and the lower ear itself, potentially resulting in pain and injury to these sensitive tissues and psychological distress. Relying on spring forces created by wedging the superior end of the scaffold-like ear-piece against the superior ridge of the concha cymba reduces the earpiece's resistance to motion-generated displacement, vibration and sheering forces produced by ordinary activities of daily living.

A necessary condition for therapeutically effective transcutaneous nerve stimulation is a securely attached electrode that is, at the same time, easily, quickly and painlessly applied and removed by a user. The need for security in the attachment coupling scheme is relative to the type of electrode, percutaneous versus transcutaneous. Unlike percutaneous electrode coupling schemes which incorporate skin piercing needles, as described by U.S. Pat. No. 9,662,269 B2, transcutaneous electrode coupling avoids the complications and risks to the user presented by a needle electrode or needle array, said complications including wound infection, pain to the user, and the need for professional attachment and re-attachment. Contemporary transcutaneous electrode-skin couplers include the use of adhesives collars surrounding the electrode and affixing it to the skin; spring-loaded clips which clasp the pinna, auricle, concha or lobes of the ear, and the use of cavity-penetrating projections inserted into the ear canal as an anchoring scheme. Each of these transcutaneous electrode-to-skin coupling schemes present potential and actual complications and challenges for a user. The user of adhesive collars is highly problematic on an uneven surface such as the human ear and the use of adhesive to secure an electrode against ear tissue and whilst withstanding gravity, motion and sheering forces may, upon removal, cause pain to the sensitive tissue of the ear of a user and require vigorous, skin irritating clean-up of the adhesive. Some manufacturers employ a spring-loaded, clip-based electrode attached to the ear lobe or to the tragus, concha or pinna of a user's ear. These ear-clip electrodes are typically attached to the stimulator device by a cable of at least twenty-five inches in length. The combined weight of the clip itself, the electrode, or electrode pair and its connecting cable necessitate the use of a clamping force sufficient to hold the clip in place against the weight of the clip and cable for the entire period of stimulation in situations where ordinary movement of the user can easily cause electrode detachment. The clamping force exerted against sensitive ear tissue for prolonged periods is a known source of discomfort to the user that can create a negative association in the mind of a user with stimulation therapy that may discourage compliance with a prescribed treatment regimen, especially when the clamping is accompanied by perceivable, slightly uncomfortable electrical stimulation. Cavity-anchoring electrodes inserted into the ear canal avoid the unpleasant clamp force of ear-clip electrodes but not the gravity drag of the cable. Such ear-canal electrodes also block the ear canal and tend to collect the waxy exudate present in the ear canal. The ear canal itself contains sensitive tissues and other structures that may be negatively affected by the insertion and wearing of inserted electrodes which plug the ear canal. One example of the ear-canal anchoring scheme is device made by Nervana® which uses the ear canal as both an anchoring structure for position maintenance and as a stimulation point. The Nervana® ear-canal plug incorporates two conductive electrodes on what is essentially an audio-emitting ear-canal plug or “bud.” A drawback with this ear-canal electrode anchoring scheme is illustrated by the fact that, according to its crowd-funding web site, Nervana LLC has received various complaints from users about “burning” sensations in the ear canal. Users of the Nervana® device are instructed to use a saline solution for conductive coupling inside the ear canal. This results in the uncomfortable presence of conductive liquid in the ear canal, which is known to loosen and mobilize ear wax which may become attached to the inserted ear electrode. Additionally, recent functional MRI (fMRI) studies have found that, among available transcutaneous auricular stimulation points, ear canal stimulation produces the lowest level of activation in key the brain areas associated with therapeutic benefits and plasticity induction: the nucleus of the solitary tract (NST) and the accumbens nucleus. Ear canal electrode placement can also produce detrimental results. The skin of the user's ear canal may suffer burns from excessive electrical current, as noted by users of the Nervana® ear-canal electrodes. This result is especially likely when a selected electrode site like the ear canal has low nerve receptivity, thus requiring higher current intensities. Repeated stimulation at relatively higher current intensities applied to the same site may produce mild burns, for example, when the ear electrode directs its energy through a metal “pole,” as does the monopolar “Ear Clip with Pole” electrode described in U.S. Pat. No. 8,457,765 assigned to and used by Alpha Stim (AKA Electromedical Products International, Inc.). The combination of small surface contact electrodes with low receptivity in targeted nerve sites virtually guarantees that higher current intensities will be required, thereby contravening Yerkes-Dodson law and raising the likelihood of electrode burns.

A stimulator device known as ElectroCore® does not include a positionable coupler, requiring instead that the user perform a coupling function. Coupling with the ElectroCore® is performed by holding the stimulator device in a precise location under the jaw, with continuous pressure against the skin to deliver the electrical signal to the targeted underlying nerve throughout the brief duration of the stimulation. In addition to the fact that many users will fail to perform this manual coupling function reliably and as instructed, users quickly weary of functioning as couplers themselves, and the tedious, unpleasant task of coupling an electrode to the skin becomes an aversive experience in its own right, resulting in poor treatment compliance which reduces treatment effectiveness.

### OBJECT AND ADVANTAGES

Transcutaneous nerve stimulation devices could produce less than optimal results for a number of reasons. The barrier of skin and tissues between the stimulation emitter (e.g., electrode) and a nerve inside the body generates strong electrical resistance which weakens the power of the electric signal delivered to the target nerve. This resistance barrier can be mitigated by using stronger electric current at the cost of producing collateral effects such as burning the skin and causing pain to the user. Most currently marketed transcutaneous auricular neurostimulation devices do not, over time, adequately maintain a constant degree of user coupler apposition to the skin, resulting in fluctuating, inconsistent and higher impedance which may reduce the degree of signal transmission through the skin, thereby reducing the strength of the signal reaching the target nerve. The security and stability of the electrode coupler are required for the positional constancy and the maintenance of conductive contact between electrode and the body of the user. Poor, inconsistent or unreliable position maintenance of the user coupler on the skin may disrupt the conductive pathway to the target nerve, causing ineffective treatment.

Most transcutaneous electrodes employed in auricular nerve stimulation are bipolar, having a positive cathode terminal and a negative anode terminal. The Nemos® device marketed by Cerbomed, for example, has two “titan electrodes” located approximately five to twelve millimeters apart on a single applicator head designed to be wedged against the superior ridge of the concha cybma. The GammaCore®, Nervana®, and NeuroSigma® stimulation electrodes follow a common scheme locating cathode and anode electrodes on the same plane, essentially side by side. The Nervana® ear-bud electrode has terminals which are both in contact with the circular wall of the ear-canal, a single plane. An obvious problem that arises with this side-by-side electrode arrangement is that the electrons emitted by the electrodes tend to follow the path of least resistance and flow between the two poles, especially since the electrical resistance of the skin is relatively high. Because of high skin resistance and electron attraction between electrodes, higher energy levels are required to transmit energy through the skin barrier into subcutaneous layers of the epidermis to reach targeted nerves. Higher energy levels can cause pain, burn the skin, and waste the limited electricity of battery-powered simulation devices.

The present invention incorporates two kinds of energy stimulation modules: the first having electrodes configured for traditional transcutaneous electrostimulation and the second having optical emitters configured for electromagnetic stimulation, a modality which takes advantage of the fact that light can be passed through the skin and its electromagnetic energy deposited in tissues including nerve fibers. In the present disclosure, both electric and electromagnetic or light energy emitters are referred to as “electrodes,” “energy emitters,” “emitters” and the like.

The electrostimulation module of the present invention includes anode and cathode electrodes on opposing sides of the ear skin, i.e., the ventrolateral and dorsolateral surfaces of the auricle, forming an electrical path between the cathode and anode terminals that passes through ear tissues and intersects the targeted nerves. This electrical intersection reduces the amount of energy required to deliver stimulation to the nerve by as much as thirty-five percent. The lower energy spend brings the intensity of electrostimulation current down below the pain threshold, reduces the likelihood of skin burns, and thereby removes obstacles for treatment compliance, namely discomfort, pain and skin burns. Additionally, recent clinical research has shown that is nerve stimulation is more clinically efficacious at lower energy levels, which is consistent with Yerkes-Dodson law. This law describes an empirical relationship between arousal (in the present case, electrical stimulation) and performance (the bodily response to or effects of stimulation) that dictates that performance (stimulation effects) increases with physiological or mental arousal (electrical stimulation), but only up to a point, beyond which more arousal (or stimulation) causes lower performance (stimulation effects). The empirical relationship described by Yerkes-Dodson law is often illustrated graphically as a bell-shaped performance curve which increases and then decreases with higher levels of arousal, in this case stimulation. High stimulation current levels may over-arouse both target nerves and the nervous system itself thereby defeating the purpose of stimulation therapy.

Single-site electrode-couplers like Cerbomed's Nemos and Nervana foreclose on the possibility of determining the most receptive nerve targets of individual users. The natural variance in the anatomic geometries of the human ear requires more flexible electrode positioning and coupling schemes which enhance fit, retention, and conductive contact among a diverse population of potential users, reduce drag and provide means for adjusting the position of emitters. For example, when a previously used location has been damaged or sensitized by excessive use, high current intensity, or compressive forces applied by the coupling means.

The invention presented herewith provides an integrated coupler-emitter array in a preferred embodiment as an ear loop, i.e., a tubular device worn seated posteriorly behind the ear within the groove space between the external ear and the head sometimes referred to as the “fold” and the “crotch” of the ear, which hereinafter shall be used interchangeable to refer to the ventral and ventrolateral dimensions and areas of the external ear or “auricle.” One of the advantages of the ear-loop design is that the weight of the cable connecting it to the signal generator is distributed to the superior arc of the loop where it rests against the top of the ear crotch, unloading the electrodes or “energy emitters” from potential disconnecting weight of cables. The ear-loop coupler also provides significant protection against the drag and sheering forces created by normal cable and body movement which can, as discussed above, exert forces that reduce consistent conductive electrode contact with the skin. The ear loop design takes advantage of the crotch between the ear and the head and dorsally near the top of the ear, which is provides a large, natural retention groove that securely anchors the ear-loop in position, even during movement of the wearer. Anchoring is further enhanced by the ear-coupler looping around from the crotch of the ear posteriorly and then descending anteriorly between the crus of helix and the tragus, and thereupon having a hub socket providing “snap-in” connection for one or more extensible, rotatable arms bearing electrodes. Fanning out from the connecting hub and having one or more points of electrode contact with the superficial ventrolateral surfaces of the ear, the one or more electrode arms simultaneously sandwich and clasp the ear between the front and rear dimensions of the ear-loop.

Each of the aforementioned prior art schemes for locating, coupling and retaining a user-attached electrode may impose limitations on the user and clinicians which reduce the effectiveness of transcutaneous stimulation of the vagus nerve. Research is still needed to pinpoint the optimal location of energy emitters, and as such research discloses new findings, improved efficacies and new methodologies, other devices designed to position electrodes at a single anatomical location may be rendered almost immediately obsolete. The modularity and flexibility of the present invention, in contrast, invites clinical research, providing an integrated platform for repeatable research and therapeutic standardization. In order to provide repeatable treatment-to-treatment consistency of nerve targeting for repeated stimulation, the electrode arms of the present invention may comprise mechanisms to mark and/or retain the positioning of electrodes relative to the anatomic dimensions of individual users. For example, extension points on the adjustable electrode arms may have millimeter hash marks, and rotational arm position may be likewise denoted by tick-marks near the socket hub.

Research is also needed to determine the most optimal and therapeutically beneficial elements of the treatment protocols for transcutaneous vagus nerve stimulation. It may soon be found, as many researchers expect, that certain electrical frequencies, waveforms, energy levels, and pulse parameters provide optimal results for different clinical entities, diseases and conditions. In addition to optimal electrode positions, there may well be optimal electrode combinations, relative to factors such as electrode size, energy deposition characteristics, electrode composition, positional location, and the like. Research on the electrical stimulation of the sympathetic nervous system continues to show effectiveness and promise in treating various is conditions such as depression, insomnia, anxiety, over-eating, addiction, obesity, inflammatory disorders, tinnitus, poor concentration, and attention deficit disorders, to name just a few.

Research has also indicated that the effectiveness of electrical nerve stimulation for disease and disorder specific therapies will likely depend on the characteristics of the electrical signal used, in terms of (inter alia) wave geometry, pulse width, use of pulse bursts, power and fluence, as well as programmed and cyclic variances in these parameters, as well as the schedule and accessibility of therapy, etc.

With regard to the signal modulation and control elements described in prior art, few if any control features are offered for the user, beyond a few presets for attenuating basic characteristics of the stimulation signal such as frequency, wave geometry, pulse-widths, etc. None of the prior art includes features required to conduct large scale research, such as a wide range of selectable or adjustable signal controls; methods to collect, track and measure user responses to stimulation through rapid symptom sampling scales and biofeedback measures; methods to access a common, internet cloud server database for storage and aggregation of user stimulation parameters, user symptom scale responses and user responses as biofeedback; methods to automate the electronic communication of user stimulation parameters and user response data to remote healthcare providers; methods enabling health care professionals to alter user stimulation setting remotely and to obtain informed consent; and algorithmic methods for adjusting and updating user stimulation parameters in accordance with emerging research findings and local user-coupler factors such as nerve field receptivity, electrode contact site conductivity, electrode position optimization, and electrode combination optimization.

Research and experience with invasive (implanted) and percutaneous stimulators has shown that generating and sustaining therapeutic levels of anti-inflammatory activity via stimulation of the parasympathetic nervous system requires between two and four hours of electrostimulation treatment daily, over a period of three months to multiple years. Translating such a treatment regime into non-invasive transcutaneous stimulation employing surface electrodes poses a variety of challenges including the fact that, for some users, repeated and/or long term electrostimulation may burn skin tissues receiving electrical current from is transcutaneous electrodes. Electromagnetic stimulation using optical emitters provides an alternative to those susceptible to electrical burns and skin reactions from microcurrent stimulation.

The electromagnetic stimulation module of the present invention includes one or more optical emitters, e.g., LEDs which may also be arranged for nerve intersection when positioned on opposing sides of the ear, i.e., the ventrolateral and dorsolateral surfaces of the auricle. Transcutaneous photo-stimulation is a nascent modality accidentally discovered by one of the inventors (Honeycutt). Electromagnetic or photo-stimulation offers unique and clinically significant advantages over electrical stimulation. Light energy passes easily through human skin and may be absorbed by targeted tissues. Light energy in the infrared band can easily penetrate up to five millimeters of skin tissue to stimulate targeted nerves in the auricular nerve field with virtually no risk of the skin burns associated with electrical electrodes. The use of photo-emitters thus enables around the clock use limited only by available power supplies.

From the foregoing, it is clear that there is a need for a system and methods that provide a flexible, adaptable, modular transcutaneous energy stimulation platform, offering multiple user-electrode coupling schemes to service the variety of nerve targets within the auricular nerve field, including the vagus nerve, the great auricular nerve, the trigeminal nerve and the lesser occipital nerve. As the interface between man and machine coupling stimulation emitters to the nerves and thereby the nervous system of the user, the electrode-skin coupling system is a most critical linkage. Weaknesses in design, functionality, flexibility, adaptability and usability of the ear-electrode system can limit the effectiveness of neurostimulation, create safety hazards such as applying the ear-electrode to the wrong ear, and create pain, skin burns, discomfort and other barriers to treatment compliance. The absence of user-response feedback, both subjective and biological, during and over the course of neurotherapy may pose a sufficiently and potentially significant risk that it should be considered a risk of unmonitored neurostimulation. Thus, a need exists to overcome the problems, limitations and challenges with the prior art systems, designs, and processes discussed above.

### DRAWING FIGURES

FIG. 1. Smart Stimulation System Block Diagram illustrates a user's human ear to which is applied an exemplar energy stimulation earpiece. Said earpiece connects to a stimulation generator package in communication with a personal computing platform. Said computing platform locally controls modalities of operation with said user and inputs biofeedback signals and serves as a gateway through the internet to a web server to enable various remote functions such as data aggregation, evaluations and operational parametric protocols.

FIG. 2A Human ear nerve fields subject to beneficial energy stimulation.

FIG. 2B Human ear ventrolateral stimulation targets,

FIG. 2C Human ear dorso-crotch stimulation targets.

FIG. 3A An exemplar adjustable stimulation earpiece to be worn by a user that provides a single ventrolateral stimulation emitter and a plurality of dorsal and a plurality of complimentary located dorsolateral and dorsal crotch electric energy stimulation emitters.

FIG. 3B An exemplar adjustable stimulation earpiece to be worn by user that provides a plurality of electric energy stimulation emitters and a plurality of complimentary located dorsolateral and dorsal crotch electric energy stimulation emitters.

FIG. 4A An exemplar adjustable stimulation earpiece to be worn by a user that provides a plurality of ventrolateral stimulation emitter and a plurality of dorsal and a plurality of complimentary located dorsolateral and dorsal crotch optical energy stimulation emitters.

FIG. 4B An exemplar adjustable stimulation earpiece to be worn by a user that provides a plurality of ventrolateral stimulation emitter and a plurality of dorsal and a plurality of complimentary located dorsolateral and dorsal crotch optical energy stimulation emitters with an audio earbud.

FIG. 5A An exemplar spring actuated adjustable compression force clip designed to be affixed to a user's ear lobe with a stimulation connection cable affixed at the bottom end of said clip.

FIG. 5B An exemplar spring actuated adjustable compression force clip designed to be affixed to a user's ear lobe with a stimulation connection cable affixed to a swivel connector at the upper end of said clip.

FIG. 5C An exemplar “press to position” ear lobe stimulation clip.

FIG. 6A An exemplar ear lobe clip version worn on a user's ear with bottom affixed connection cable looped over said ear.

FIG. 6B An exemplar ear lobe clip version worn on a user's ear with upper affixed connection cable looped over said ear.

FIG. 6C An exemplar ear lobe clip version worn on a user's ear with bottom connection cable attached to a separate ear loop.

FIG. 6D An exemplar energy stimulation earbud designed for ventrolateral coupling contact.

FIG. 6E Illustrates an exemplar energy stimulation earbud in user's ear with contact and cable.

FIG. 7. Illustrates a configuration of a user wearing a stimulation electronics package supported by a lanyard about the neck with attached cable leading from said lanyard to ear worn stimulation energy emitter coupling module assembly.

FIG. 8 Remote Function Block Diagram illustrates the signal and information flows from a user's mobile computing platform, through the internet cloud, web server remote function application and health care providers.

FIG. 9 Stimulator Unit Block Diagram illustrates the major functional components incorporated in a typical stimulation generator electronics package.

### REFERENCE NUMERALS IN DRAWINGS

80 Human ear,
81 Trigeminal nerve fiber zone
82 Vagus nerve fiber zone
83 Great auricular nerve fiber zone
84 Lesser occipital nerve fiber zone
85 Ventrolateral Trigeminal Nerve (v.3) (TNV3) Target D1
86 Ventrolateral Auricular Branch Vagus Nerve (ABV) Target D2
87 Ventrolateral Lesser Occipital Nerve (LON) Target D3
88 Ventrolateral Auricular Branch Vagus Nerve (ABV) Target D4
89 Ventrolateral Great Auricular Nerve (GAN) Target D5
90 Dorsolateral Trigeminal Nerve Target V1
91 Dorsolateral Auricular Branch Vagus Nerve Target V2
92 Dorsolateral Lesser Occipital Nerve Target V3
93 Dorsolateral Auricular Branch Vagus Nerve Target V4
94 Dorsolateral Great Auricular Nerve Target V5
100 Energy emitter coupling module assembly
101 Ear loop structure
102 Ear loop dorsal lateral emitters
103 Ear loop crotch emitters
104 Energy stimulator ventrolateral contact shape 1
105 Ear loop arm swivel assembly
106 Energy stimulator ventrolateral contact shape 2
107 Ear loop swivel arm
108 Ear loop extension arm
109 Ear stimulator connection cable
120 Optical energy ear loop
121 Optical energy dorsal lateral emitter
122 Optical energy crotch emitter
130 Optical energy ear loop with audio
131 Audio swivel hub subassembly
132 Audio earbud speaker subassembly
140 Stimulator package assembly
141 Stimulator lanyard
150 Ear lobe compression type clip assembly
151 Ear lobe clip arm 1
152 Ear lobe clip arm 2
153 Ear lobe clip arm cam slot
154 Ear lobe clip arm cam slider
155 Ear lobe clevis position lock and release assembly
156 Ear lobe arm compression torsion spring
157 Ear lobe stimulation emitter contact
158 Ear lobe clip connection cable
160 Ear lobe press to set position clip assembly
161 Ear lobe clip cable connector swivel
162 Ear lobe clip crotch loop
163 Conductive adhesive ear lobe energy emitter coupler
164 Ear lobe clip press to set position release
170 Earbud wedge
171 Earbud concha loop
200 Stimulation electronics unit
300 Personal mobile computing platform

### DESCRIPTIONS OF PREFERRED AND SYSTEM EMBODIMENTS

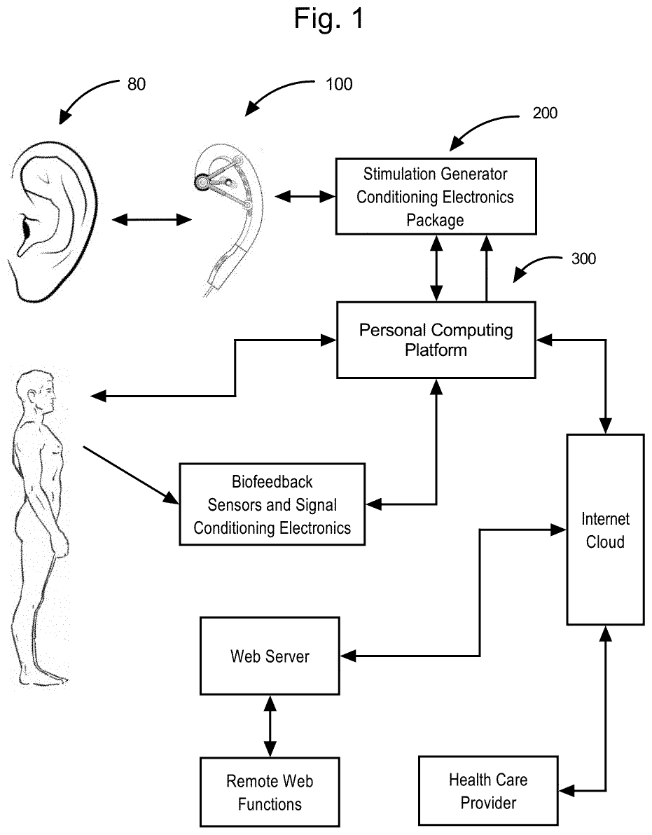

The present invention comprises a system of hardware components and software integrated to provide energy stimulation to specific nerve targets proximal to a human user's ear. As illustrated by the system block diagram in FIG. 1, said components include a personal mobile computing platform 300, a stimulation unit 200 and a variety of electromagnetic energy stimulation emitter earpieces 100 worn about the ear 80.

A significant advantage of the present invention is that stimulation of a target nerve field is by means of energy flow through the nerve field rather than conventional electric stimulation techniques in which the electric current flows between two adjacent surface contacting electrodes.

For clarification, various embodiments for each component and methods for use are categorized and described separately herein.

User Mobile Personal Computing Platform

A preferred embodiment for said computing platform 300 consists of a conventional smart phone, tablet or computer, providing an intelligent graphic user interface (GUI) with Internet and local connectivity network interfaces such as WIFI, Bluetooth and USB. Said controller operates under software applications to communicate with a with a cloud based web server for client data tracking, software upgrades and user session protocol stimulation optimization. The controller communicates with said stimulation unit 200 by means of wireless connection such as Bluetooth or a wired serial communication such as USB.

In a further embodiment, said computing platform incorporates audio output to instruct and prompt the user to invoke proper control commands. Such audio instructions include proper ro attachment of the stimulation emitter and prompts for setting stimulation parameters and protocol selection. Said instructions may be downloaded remotely from health care providers via the internet to the user mobile computing platform.

In a further embodiment, said computing platform incorporates voice recognition to enable the user to conveniently invoke control commands such as to start, pause or end a stimulation session and to adjust the stimulation intensity level.

In a further embodiment, said computing platform provides a graphical user interface to enable control output stimulation parameters including waveforms, intensity levels, frequency, as well as selection of pre-programmed protocols.

In a further embodiment, said computing platform may be used to output selected music to be received by said stimulation unit by means of wireless transmission or by direct wired audio output. Said audio output music is processed by said stimulation unit electronics to modify and coordinate various energy stimulation output waveforms, intensity and frequency parameters. Said stimulation output as modified by music may enhance the efficacy of energy stimulation for said user.

Stimulation Unit

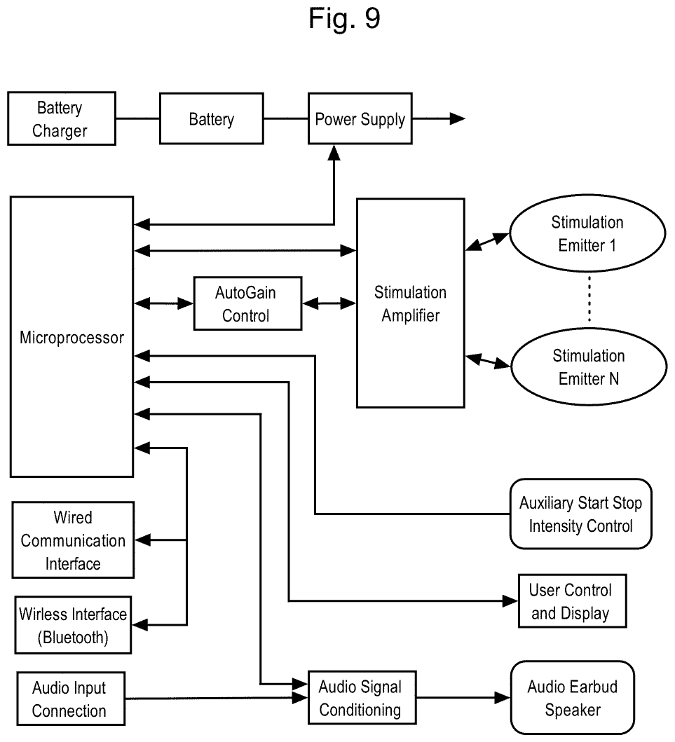

A preferred embodiment for said stimulation unit 200 includes electronic circuitry and battery powered, microprocessor controlled, multichannel amplifier system as depicted in FIG. 9. Said stimulation unit communicates with said user computing platform 300 unit by means of wireless connection such as Bluetooth or using a wired serial communication such as USB. Stimulation signals including waveforms, frequency and amplitude are generated by said microprocessor and converted to analog output energy by means of amplifier electronics.

A further embodiment, wherein the output stimulation energy is electric current, includes electronic circuitry and software to monitor output voltage and current and automatically adjust output to maintain stimulation setpoint levels in order to compensate for impedance variations inherent in maintaining consistent electrical contact between emitter contacts and associated skin tissue targets.

A further embodiment, wherein the output stimulation energy is electric or electromagnetic (photonic/optical) includes electronic circuitry and software to generate various ro waveforms such as square, pulsed, triangular and sinusoidal. Further, in the case of electrical said output stimulation energy, the intensity level is ramped up in a manner to minimize transient transmission cable inductive spikes inherent with square waves that has been shown to irritate skin tissue with prolonged use.

Current research results indicates the optimal effect of nerve stimulation occurs in the range from 0.5 to 250 Hertz. Additionally, research also has found electromagnetic energy provides optimal parasympathetic nervous system response in the range of wavelengths from 400 to 1600 nanometers with a fluence power density from 0.5 to 35 joules per square centimeter.

A further embodiment incorporates electronics and application software to modify said stimulation signals such as waveforms, intensity and frequency modified in accordance with audio input signals such as music received from said computer platform by through wireless means such as Bluetooth or hardwired connection.

A further embodiment provides electronic circuitry to enable use of a rechargeable battery to provide power to the stimulation unit.

A further embodiment utilizes the microprocessor to monitor and control power supply and battery charger functions and communicate battery condition data to said computing platform.

A further embodiment provides wired or wireless connection of an auxiliary remote control device connected to said stimulation unit to enable basic control functions such as start, stop, pause and intensity control. Said control device may also include rudimentary operational displays as convenient to allow operation without the need for realtime connection to said user mobile platform.

Energy Stimulation Emitters

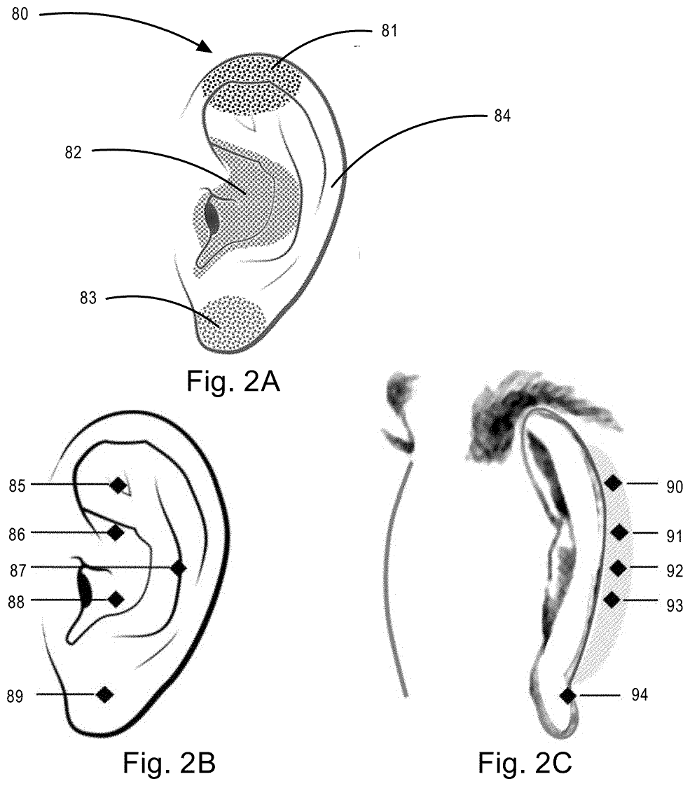

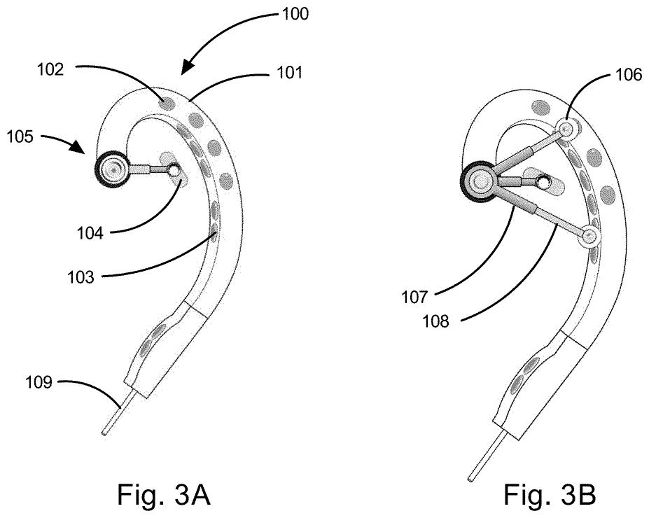

Embodiments for said energy stimulation emitters may include electrical or photonic types designed to target specific nerve field targets as indicated in FIG. 2A, FIG. 2B and FIG. 2C and held proximal to a user's ear by means of energy emitter coupler apparatus. FIG. 3A illustrates an electrical energy type with at least one emitter 104 supported by an ear loop 100 assembly fitted over and within the fold of the ear and skull, termed herein as crotch.

FIG. 3B illustrates an embodiment incorporating an ear loop 101 integrating one or more electrical energy coupling emitters 102 and 103 designed to contact the dorsal side nerve targets 90 through 93 as depicted in FIG. 2C. A rotary slip ring swivel assembly 105 supports one or more swivel arms 107 and mating extension arms 108. Said rotary slip ring provides mechanical coupling and may be designed utilizing a removal pin or post, or by plug-in features made part of said swivel arms. Additionally, said rotary slip ring can include electrical contacts fabricated by means of mechanical components molded or assembled into, or by means of conductive material molded or printed as part of said swivel. Said extension arm supports electrical energy emitters 104 or 106 configured to address specific desired said nerve targets. As indicated, said emitter 104 is shaped to optimally fit and contact the Ventrolateral Auricular Branch Vagus Nerve target 88 as depicted in FIG. 2B. FIG. 3B illustrates an embodiment of said ear loop with said slip ring swivel assembly supporting three said support arms with different shaped energy coupling emitters. Said emitter 106 is shaped to optimally fit and contact the Ventrolateral Trigeminal Nerve (v.3) (TNV3) 85 and the Ventrolateral Auricular Branch Vagus Nerve (ABV) 88.

In one embodiment, said ear loop connects to said stimulation unit by means of a multiconductor electrical cable 109. In a further embodiment, said ear loop includes stimulation electronics and communicates with said user controller via wireless or by means of said cable. This embodiment may also include said cable 109 to provide electrical power and utilize fiber optic for transmission of stimulation waveforms to said loop integrated stimulation electronics.

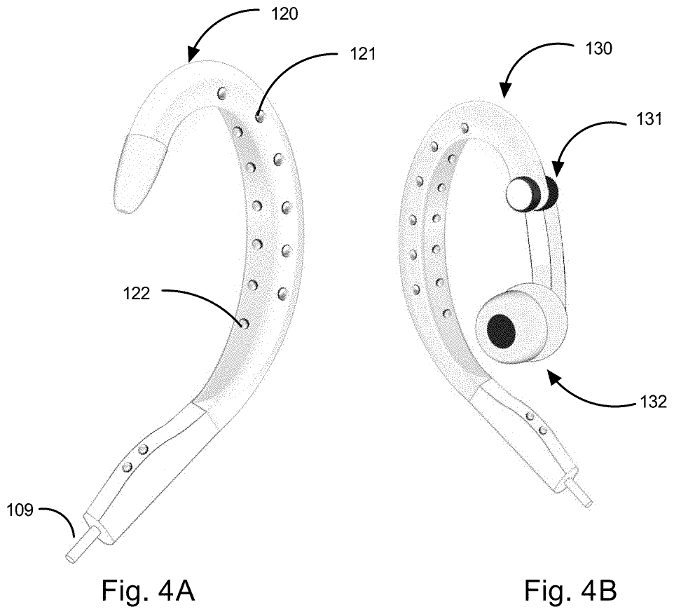

Further embodiments in FIG. 4A illustrates an optical energy stimulation ear loop 120 with an array of optical dorsal lateral energy emitters 121 and an array of optical crotch energy emitters 122. FIG. 4B similarly illustrates said loop and emitter arrays with addition of an electromechanical swivel 131 and audio earbud 132 to operate in coordination with stimulation electronic circuits and software to enable modulation of the intensity, frequency and waveforms of stimulation energy with the fundamental frequency of selected audio signals which may be music, biofeedback audio response or other sounds found therapeutically beneficial.

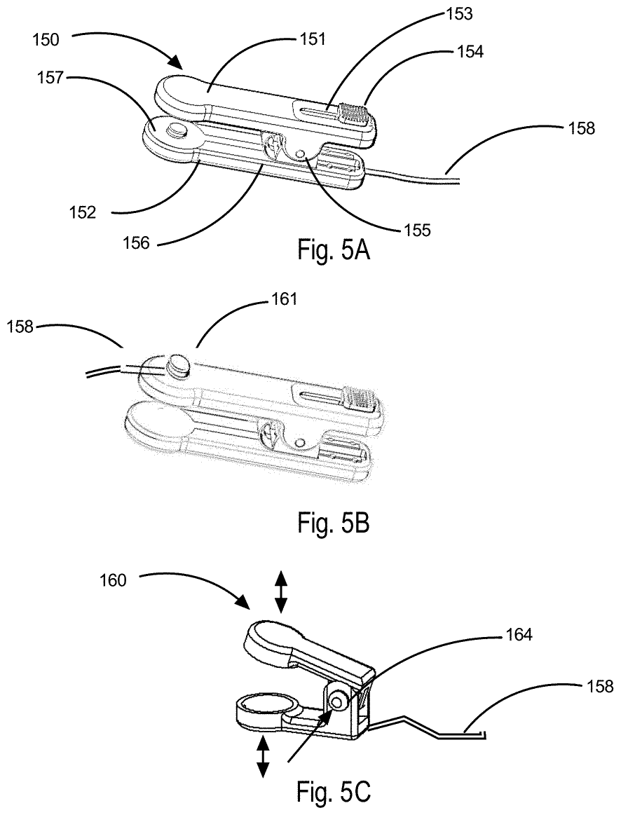

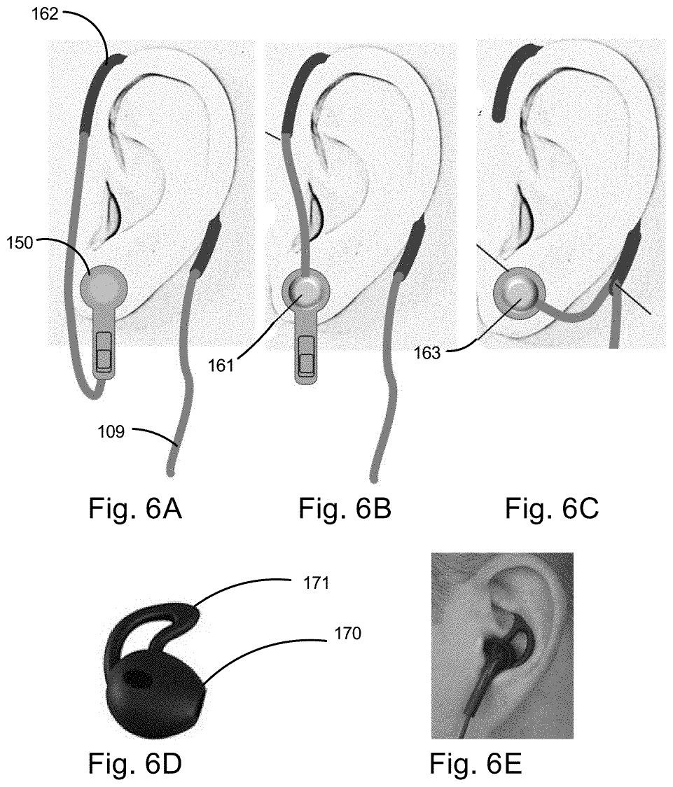

Further embodiments in FIG. 5A FIG. 5B, FIG. 5C, FIG. 6A, FIG. 6B and FIG. 6C provide various means for electromechanical connection for an emitter coupling contacts on said user ear lobe. FIG. 5A and FIG. 5B illustrates a spring compression clip type 150 whereby the compression force exerted by a torsional spring 156 squeezing upon the earlobe by the ear lobe stimulation emitter contacts 157 as exerted by ear lobe arm 1 151 and ear lobe arm 2 152 is ro adjustable by means of a manually operated cam slider 154 restrained by a cam slot 153. Said cam and slot include features such as frictional grooves or ratchet teeth in order to maintain user set position. Said torsional compression spring may be a metal spring component or conveniently molded or 3D printed as part of one or combined said arms. FIG. 5C illustrates a further embodiment of a press to set position earbud clip 160 whereby said coupling emitters are conveniently pressed into a set position upon the ear lobe by the user instead of exerting an active spring compression as is typical. The set position is maintained by means of friction or ratchet features integrated into the ear lobe clevis position lock and release assembly 155 which includes a torsional release spring 164 actuated to open said arms when depressed. Said torsional release spring may be a metal spring component or conveniently molded or 3D printed as part of one or combined said arms. The embodiment of FIG. 5B illustrates a cable connector swivel 161 which permits connection cable 158 to be conveniently connected toward the emitter. This feature may be applied to each of the three embodiments shown in FIG. 5A, FIG. 5B and FIG. 5C.

FIG. 6A, FIG. 6B and FIG. 6C illustrates further embodiments of ear lobe clips as worn on said user's ear. FIG. 6A shows a configuration wherein the emitter coupling 150 is attached to an ear lobe crotch loop 162 which connects to stimulator cable 109. FIG. 6B similarly illustrates an ear lobe clip 161 with said swivel connection. FIG. 6C similarly illustrates emitter coupling connection to said crotch loop and said cable by means an adhesive type ear lobe attachment 163 to support either an electrically conductive or photonic/optical emitter contact. FIG. 6D and FIG. 6E illustrate a typical ear wedge 170 and earbud concha loop 171 devices similar to conventional and available audio earbud speakers, the design of which may be employed to conveniently secure said emitters within the concha.

In a further embodiment, said loop supports the attachment of at least one modular, interchangeable emitter arm assembly 105 supporting a swivel arm 108 and extension arm 108. Each emitter arm thereby supports an interchangeable modular emitter contact. Each modular s arm integrates swiveling electromechanical connections for attachment to said loop and emitter. Said attached arms may be of a specific length, or incorporate a telescoping feature in order to conveniently accommodate and optimize positioning of said emitter(s) for a particular user.

In a further embodiment, said swivel and arms provide indexing features to indicate user set swivel and extension positions to conveniently aid the user to record and reset said positions for future use. A further embodiment provides the means to lock said set positions.

The present invention includes a clip type emitter coupling to enable positioning energy emitter contact upon a user's ear lobe in either or both anteriorly or posteriorly. In order to alleviate user discomfort experienced by conventional clips, the present invention provides two different clip design versions as illustrated in FIG. 5A and FIG. 5B. One version incorporates a sliding cam 154 that enables the user to adjust the spring compression pressure squeezing emitter contacts upon the ear lobe. FIG. 5C incorporates a spring that causes the emitter contacts to open away from the ear lobe. The user then presses the jaws to close against the ear lobe with sufficient pressure to firmly yet comfortably hold the clip in position for effective emitter contact.

Said ear loop, arms and emitter contacts may be manufactured using conventional plastic injection molded plastic technology. The design may also be conveniently utilize new and future 3D printing technology that can incorporate electrically conductive traces and electrode contact pad surfaces in order to minimize manufacturing process steps, manual assembly and number of components. One advantage of 3D printing wearables is the potential for combining with 3D body (dimension) scanners to make individually customized, fit-optimized wearables that are consequently capable of reading biosignals and delivering stimulation at the lowest effective fluence thereby reducing potential nerve and tissue damage.

Stimulator Unit Wearable Devices

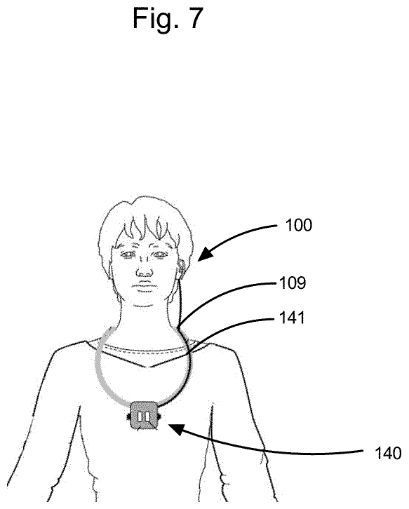

FIG. 7 depicts a stimulator package assembly 140 to be worn by the user fastened to a lanyard 141. Said stimulator package assembly includes said stimulator unit which may be connected by a cable 109 or wireless to said energy emitter coupling module assembly 100. Further embodiments to enable the user to conveniently wear said stimulator package include strap clip, pocket clips, belt clip, or other typical fastening devices such as adhesive or hook and loop that provide wearing about the neck, limbs or about the body. In addition, said wearable means may also be used to fasten or attach biofeedback sensors.

Web Based System

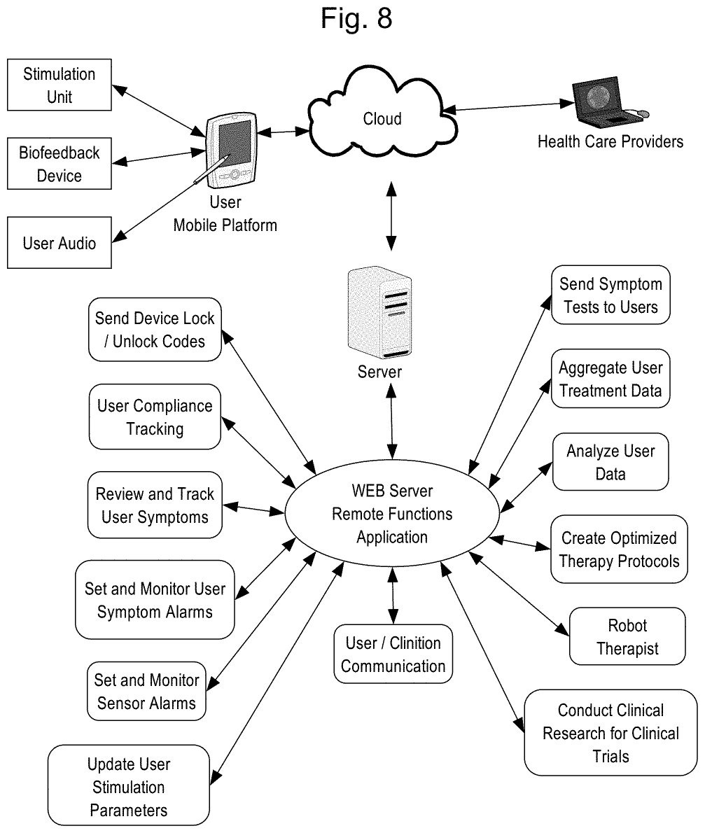

FIG. 8 depicts an integrated system with a user mobile platform to provide communication via the internet cloud with remote health care providers and web server hosting remote function application software. Said user mobile platform provides local and internet wireless communication, graphical user interface and computational capability and serves to monitor and control said stimulation unit, biofeedback devices and audio input and output. Said web server and application software provide a variety of services for user by users and by health care providers, clinicians and robotic therapist. Application functions include sending user device lock and unlock codes (password) for user security; user compliance tracking; review and tracking of user system parameters; setting and monitoring of user symptom alarms; updating user stimulation parameters; sending of symptom tests to users; storage and aggregating user treatment data; analysis of user data; developing optimized therapy protocols; data formatting for clinical research for clinical trials.

Manufacturing Techniques

Devices described by the invention including the ear loop and ear lobe clip embodiments as described and illustrated can be manufactured utilizing conventional materials and processes such as single and multiple plastic injection molded parts, overmolded plastic and silicone and electrically conductive silicone metal stamped electrical and mechanical components and assembled using bonding adhesives. Electrical connections and signal routing within said devices can be readily manufactured utilizing direct wiring; for example between said emitter contacts and electrical cable connections. It is further anticipated that said devices can advantageously be designed and fabricated using advanced materials and additive manufacturing (AM/3D printing) processes, especially as technology improves and costs are reduced. Significantly, components can be manufactured in one process step, utilize a variety of materials to provide integrated electrically conductive circuits, selective flexibility/rigidity and colors, and rapid and one of a kind customization.

A current prototype design of said three arm earloop as described and illustrated in FIG. 3D, for example, enables 3D printing of all mechanical components including the electrical cable connection to cable 109; internal electrically conductive traces and electro-mechanical slip ring contacts at 104 and 105 as well as the telescoping swivel and extension arms 107 and 108; and electrically conductive nerve target said emitter contacts 102, 103, 104 and 106.

1. An energy stimulation therapy system for delivering, controlling and monitoring energy stimulation applied transcutaneously to the body of at least one user, comprising
an electronic stimulator package comprising stimulation signal generating electronics, signal conditioning and control electronics, and stimulator monitoring electronics, and at least one channel of stimulation energy output;
a power source with power modulation electronics and battery recharging circuitry;
electronic hardware and software for communication with at least one computer device;
said stimulator conditioning and control electronics configured to produce the selection and control of at least two stimulation parameters belonging to a group of stimulation parameters that includes power amplitude, fluence, waveforms, wavelengths, pulse widths, phase characteristics, stimulation channels, stimulation frequencies, stimulation session periods, time intermittency and intervals of stimulation delivery and the like, and compilations thereof;
at least one energy emitter module comprising a coupling apparatus and at least one energy emitter configured for removably coupling said at least one energy emitter module to the body of a said at least one user, with said coupling structure having between 0.1 and 84 grams of coupling compression force against the skin of a said at last one user.

2. The energy stimulation therapy system according to claim 1 further comprising
electronic switches for powering on and powering off said stimulator package, for selecting and enumerating values of said stimulation parameters, for selecting grouped stimulation parameters called “protocols” for accepting user input and for performing program selection and control operations, and the like;
a computerized graphical user interface configured to selectably display the operational status of said stimulator package, selectable programs of stimulation called “protocols,” power and battery levels, selectable time periods, selectable said stimulation parameters and the like;
at least one energy emitter of said at least one energy emitter coupling module is selected from a group of energy emitters that includes emitters of electrical energy, and optical emitters of electromagnetic energy, and acoustic emitters of sound energy;
control, conditioning and switching electronics for selecting and controlling a plurality of stimulation energy modalities employing energy variants according to the type of energy emitted by selected said energy emitters.

3. A wearable energy emitter module comprising
at least one energy emitter
at least one ear-worn loop coupler designed to be worn looping from behind the human ear over the superior crotch of the ear extending forward ventrally and then inferiorly to a position superiorly located above the tragus;
said at least one ear-worn loop coupler designed to couple said at least one energy emitter to the external ear tissue of a said at least one user;
said at least one ear-worn loop coupler having a weight between 2.5 and 84 grams;
said at least one energy emitter selected from a group of energy emitters that includes emitters of electrical energy and emitters of electromagnetic energy;
said at least one energy emitter having physical contact with the external ventral and ventrolateral skin surfaces of the auricle and pinna, and particularly the conchal bowl, concha cymba and tragus of the ear of a said at least one user.

4. The wearable energy emitter coupling module according to claim 3 wherein said energy emitter coupling module composed as said wearable said at least one ear-worn loop coupler, further comprising
a mounting socket composed on the superior forward end of said at least one ear-worn loop coupler about its terminal position above the tragus;
said mounting socket composed as a bearing and coupling structure designed for the removable electromechanical connection of at least one adjustable arm;
at least one adjustable arm having adjustability features selected from a group of adjustability features including movability, rotatability, length extension and contraction, torsionability, flexibility, bendability and the like;
at least one energy emitter located on said at least one adjustable arm configured to deliver energy to the body of a said at least one user;
said mounting socket composed to conduct electricity from said wearable said at least one ear-worn loop coupler to said at least one energy emitter located on said at least one adjustable arm;
conductive material comprising the electromechanical connection of the said at least one adjustable arm to said mounting socket selected from a group of conductive materials that includes electrically conductive metal wire, electrically conductive metallic tracing, electrically conductive filaments, metal plating, 3-D printed conductive material, conductive inks, and the like, and combinations thereof;
said mounting socket is further composed as a snap-in port for removably connecting said at least one adjustable arm to said at least one ear-worn loop coupler.

5. The wearable energy emitter coupling module according to claim 4 wherein said energy emitter coupling module composed as said wearable said at least one ear-worn loop coupler, further comprises
said at least one energy emitter coupled to the ventral surface of the stem of the said at least one ear-worn loop coupler thereby having coupling contact with the skin of the dorsolateral ear crotch and dorsal surfaces of the auricle and pinna comprising said at least one user's external ear;
selectable energy emitter coupling modules belonging to a group of energy emitter coupling modules that includes arm-mounted energy emitter coupling modules, clip-mounted energy emitter coupling modules, energy emitter modules mounted on spring-tensioned apparatuses and torsion-adjustable spring apparatuses configured to enable mounting of energy emitters, and an adhesively mounted conductive-gel energy emitter module;
said at least one adjustable arm is additionally composed to be length adjustable having said length adjustability in the range from 5 to 85 millimeters;
at least one electrical slip-joint rotary connector fastening said at least one adjustable arm to said mounting socket, with at least one electrical slip-joint rotary connector having at least one electrically conductive circumferential slip ring for electronic communication between said ear-worn loop coupler and said at least one adjustable arm;
said mounting socket is further composed having, within the interior surface of said mounting socket, at least one electrically conductive ring structure designed to be in physical contact with the said at least one electrically conductive circumferential slip ring located on said least one electrical slip-joint rotary connector.

6. The wearable energy emitter coupling module according to claim 5 wherein said energy emitter coupling module composed as said wearable said at least one ear-worn loop coupler, further comprises
said at least one ear-worn loop coupler may be composed with anatomically differentiating structural features corresponding to the left ear of said at least one user;
said at least one ear-worn loop coupler may be composed with anatomically differentiating structural features corresponding to the right ear of said at least one user;
a plurality of said adjustable arm modules, each having at least one said energy emitter;
multiple said adjustable arms simultaneously connected to said mounting socket by incorporating with each said adjustable arm said at least one electrical slip-joint rotary connector, wherein the said electrical slip-joint rotary connectors on said multiple adjustable arms maybe be nested within the electrical slip-joint rotary connectors on other said adjustable arms and within said mounting socket, with each said conductive circumferential slip ring and said socket port ring structure pair comprising at least one said channel of electrical conductance electronically linking said energy emitters on said adjustable arms with said at least one ear-worn loop coupler;
said at least two energy emitters located on said at least one ear-worn loop coupler spatially arranged to have contact on the opposing, contralateral ventral and dorsal surfaces of the auricle to produce energy emissions designed to intersect nerve targets located between said ventral, ventrolateral and dorsal, dorsolateral surfaces, with said nerve targets belonging to a group of nerve targets within the auricular nerve field.

7. The energy stimulation therapy system according to claim 2 further comprising
at least one computer device selected from a group of computer devices that include a conventional desktop computer, a notebook computer, a laptop computer, a smartphone, a tablet, a handheld computer and a wearable, user-attached computer device;
hardware and software for wired and wireless electronic communication between said at least one computer device and said stimulator package;
communication hardware and software installed on said at least one computer device to enable internet connectivity and the communicative exchange of data with at least one remote server;

8. The energy stimulation therapy system according to claim 7 further comprising a method of optimizing user control of stimulation parameters wherein
a said at least one user selects stimulation parameter settings by at least one communicative action selected from the group of communicative actions that includes audible communication, touch-based communication on a touch-sensitive device, switch-based communication using switches;
software residing on said at least one computer device includes at least one algorithm for ramping up said stimulation parameters from a base level in a series of selected increments of said stimulation parameters, particularly stimulation intensity and frequency;
software residing on said at least one computer device having at least one algorithm for ramping down said stimulation parameters from a base level in a series of selected decrements of said stimulation parameters, particularly stimulation intensity and frequency;
at least one algorithm which executes at least two stimulator control functions belonging to a group of stimulator control functions that includes powering said stimulator package on and off, starting, pausing and stopping a stimulation session, increasing and decreasing stimulation intensity, increasing and decreasing stimulation frequencies, and the like;
a microphone circuit in electronic communication with said computer device and said stimulator package enabling a said at least one user to configure optimal stimulation parameters via voice commands;
at least one algorithm for processing user verbalized commands, said stimulation parameters and keywords received by said microphone circuit.

9. The energy stimulation therapy system of claim 7 further comprising software installed on a said at least one computer device having
at least one database for storing data comprising stimulus queries and statement stems used in psychological, behavioral, emotional, symptomological and experiential tests, surveys, questionnaires, rating scales and the like, designed to query said at least one user regarding user experience matters belonging to a group of user experience matters that includes the nature, type, frequency, duration, and intensity of said emotions, symptoms, experiences and related manifestations and measures of psychological, emotional, and bodily conditions, disorders, and diseases, including the mobility, health and fitness, activities and social life of said at least one user;
at least one database for storing data comprising the responses of a said at least one user to said stimulus questions and statement stems;
at least one database for storing data comprising communications exchanged between a said at least one user and at least one remote therapist, wherein said communications may include conversations between a said at least one user and a said remote therapist and said stimulation parameters to parameterize and thereby control and modify the operation of a said at least one user's said stimulation package;
at least one software algorithm to monitor and track the time, date and duration of a said at least one user's stimulation sessions and the stimulation parameters used, wherein the said at least one software algorithm may be configured to present reminder advisements to the senses of a said at least one user regarding stimulation according to a recommended schedule of stimulation frequency, duration and stimulation parameters, including algorithm-developed schedules;
said at least one software algorithm to monitor and track the time, date and duration of a said at least one user's stimulation sessions additionally transmits such data to a said at least one remote server.

10. The energy stimulation therapy system of claim 9 further comprising
server software composed to produce an encryption-secured, access-controlled web-based graphical user interface configured to remotely and selectably monitor, display and control said stimulation parameters of a said at least one user's said stimulator package via said internet connectivity of said at least one user's said at least one computer device;
at least one database configured to store the data of a said at least one user belonging to a group of data that includes user account data, user medical history data, said stimulation parameters, said user stimulus-response data, said biofeedback sensor data, said user-reported symptoms and the like;
at least one software algorithm to monitor and track the time, date and duration of a said at least one user's stimulation sessions and the stimulation parameters used;
at least one database configured to store data comprising stimulus questions and statement stems used in psychological, behavioral, emotional and experiential tests, surveys, questionnaires, rating scales and the like;
at least one database configured to store data comprising responses of a said at least one user to said stimulus questions and statement stems used in psychological, behavioral, emotional and experiential tests, surveys, questionnaires, rating scales and the like;
at least one database configured to store data comprising data received from biological sensors coupled to the body of a said at least one user;
at least one database configured to store data comprising said communications exchanged between said at least one user and said at least one remote therapist;
at least one software-encoded algorithm to collect, aggregate and analyze said stimulus-responses of a said at least one user to said stimulus questions, said biological sensor data and said personal information of said at least one user collectively called “user data;”
at least one software-encoded algorithm to develop from said user data optimized stimulation parameters, optimized stimulation therapy and robotic stimulation therapy protocols;
at least one algorithm providing remote monitoring and analysis of said user data;
at least one algorithm providing remote control functions designed for use by at least one remote therapist selected from a group of remote therapists that includes human paraprofessionals, human healthcare professionals and at least one robotic therapist comprising at least one artificial intelligence algorithm programmed in software;
at least one algorithm enabling at least one remote therapist to select, apply and transmit said stimulation parameters and protocols to the said at least one computer device of a said at least one user for configuring and controlling the stimulation produced by a said at least one user's said stimulator package.

11. The energy stimulation therapy system of claim 10 further comprising a remote, server-based graphical user interface control panel programmed and configured to comprise
server software configured for a said at least one remote therapist to select floor and ceiling threshold values for said biofeedback sensor data, said stimulus-response data, said personal information data and said stimulation parameter data received from said at least one users' computer device, to serve as decision-points;
server software comprising said at least one algorithm using said floor and ceiling threshold values as trigger-points for the automatic generation of alarms, notices, and automatic responses sent to a said at least one user's computer device, and to selected other parties and devices;
server software configured for the transmission of said stimulation parameters selected by a said at least one remote therapist to the said at least one computer device of a said at least one user.

12. A wearable energy emitter coupling module comprising at least one energy emitter circuit mounted on a clip-like coupler having two opposing, elongated jaw-like surfaces wherein
said at least one energy emitter is selected from the group of energy emitters that includes metallic electrodes, optical emitters, graphene emitters, conductive filament, conductive ink and the like;
said two opposing elongated jaw-like mounting surfaces may be composed with a positional biasing mechanism selected from a group of positional biasing mechanisms that includes a leaf spring, spring steel, a coil spring, an active hinge, a compressible elastomeric body and the like;
said clip-like coupler includes a coupling-locking mechanism selected from a group of coupling-locking mechanisms that includes a static lock, friction-lock, a locking cam, ratchet, friction ridges and the like, maintains a user-set separation distance between said two opposing elongated jaw-like structures;
electrically conductive pathways are composed of conductive compositions belonging to a group of conductive compositions that includes screen-printed carbon ink, 3-D printed conductive filaments, metallic tracing, metal wires, and screen printed metal inks and the like.

13. The energy stimulation therapy system according to claim 2 wherein said at least one energy emitter module comprises a wearable electromagnetic energy emitter coupling module having at least one optical emitter in electronic communication with said stimulator package, wherein
said wearable optical electromagnetic energy emitter coupling module is configured to be worn proximally to the external surfaces of the human ear comprising the auricular nerve field.

14. The wearable energy emitter coupling module according to claims 3, 4, 5, and 6 wherein said at least one ear-worn loop and energy emitter coupling module may further comprise
stimulation generation electronics and stimulation control, modulation and switching electronics having wireless electronic communication with a said at least one user's said computer device;
a power source and battery recharge circuits wherein said power source is selected from a group of battery power sources that include alkaline batteries, lithium batteries, capacitor batteries, micro-batteries and the like.

15. The energy emitter coupler modules of claims 2, 3, 4, 5, 6, 12 and 14, wherein
said at least one energy emitter may be comprised of an electrode circuit having both negative and positive terminals configured to deliver electrical stimulation to the body of a said at least one user;
said at least one wearable energy emitter coupling module includes at least two energy emitter units spatially arranged to have contact on the opposing, contralateral ventral and dorsal surfaces of the auricle to produce energy emissions designed to intersect nerve targets located between said ventral, ventrolateral and dorsal, dorsolateral surfaces, with said nerve targets belonging to a group of nerve targets within the auricular nerve field.

16. The energy emitter coupler modules of claims 2, 3, 4, 5, 6, 13, 14 and 15 wherein said at least one energy emitter may be comprised of an emitter of electromagnetic energy, wherein
said at least one emitter of electromagnetic energy is selected from a group of optical emitters that includes LEDS, OLEDS, VCSELS, optical graphene emitters and the like;

17. The energy stimulation therapy system including claims 2, 3, 4, 5, 6, 7, 8, 9, 10 and 15 wherein
said energy stimulation comprises electrical energy stimulation delivered to the body of a said at least one user via said at least one electrical energy emitter module coupled to the body of a said at least one user;
said electrical stimulation current is comprised having at least one electrical frequency selected from a range of electrical frequencies between 0.5 hertz to 250 hertz;
said electrical current is comprised having a waveform selectable from a group of waveforms that includes sinusoidal waveforms, triangular waveforms, square waveforms, and combinations thereof and the like.

18. The energy stimulation therapy of system of claims 2, 3, 4, 5, 6, 7, 9, 10, 11, 13, 14 and 16 wherein
said energy stimulation comprises electromagnetic energy stimulation delivered to the body of a said at least one user via said at least one electromagnetic optical energy emitter module coupled to the body of a said at least one user;
said at least one emitter of electromagnetic energy is configured for emitting and transcutaneously delivering to the body of a said at least one user electromagnetic energy having wavelengths selected from a range of wavelengths between 400 and 1600 nanometers;
said electromagnetic energy is delivered to the body of a said at least one user having a power density selected from the range of fluence between 0.5 and 35 joules per square centimeter.

19. The energy stimulation therapy system according to claims 7, 9, 10, 11, 17 and 18 further comprising
at least one biofeedback sensor module removably coupled to the body of said at least one user to enable monitoring of a said at least one user's biological signals and status;
communication hardware and software protocols electronically linking said biofeedback sensor module with a said at least one user's said at least one computer device, wherein said communication hardware and software protocols are selected from a group of communication hardware and software protocols that includes Bluetooth, Wi-Fi, Zigby, and the like;
said at least one sensor in said biofeedback sensor module belonging to a group of biofeedback sensors that includes a heart rate sensor, a Heart Rate Variability (HRV) sensor, a blood pressure sensor, an oxygen saturation sensor, a breathing sensor, a sensor for detecting peripheral vasodilation and vasoconstriction, sensors for detecting autonomic nervous system activity, sensors for detecting brainwaves and the like;
software installed on said at least one computer device including at least one database for storing data received from a said at least one user's said biofeedback sensor module.

20. The energy stimulation system of claims 2, 3, 4, 5, 6, 17 and 18, wherein said stimulator package additionally comprises
at least one audio input channel;
frequency analysis electronics and an algorithm to determine the fundamental dominant frequency of incoming audio received via said at least one audio input channel selected by a said at least one user from a group of incoming audio that includes voice audio, music audio and audio ambient in a said at least one user's immediate physical environment;
frequency modulating and conditioning electronics which modulate at least one stimulation signal according to the said determined dominant frequency of said input audio;
at least one audio emitter affixed to the descending dorsal stem of said at least one ear-worn loop coupler wherein said at least one audio emitter is positioned and worn proximal to at least one ear-canal of a said at least one user.
